SEQUENCE LISTING (22 sequences)

SEQ ID NO: 1 - protein, 448 aa, synthetic construct
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 2 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNK

SEQ ID NO: 3 - DNA, 1344 bp, synthetic construct
  source 1..1344: mol_type = other DNA
atgccatttgttaataaacaatttaattataaagatcctgtaaatggtgttgatattgcttatataaaaattccaaatgtaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataag

SEQ ID NO: 4 - DNA, 1344 bp, synthetic construct
  source 1..1344: mol_type = other DNA
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacctttacaaaccctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacagccagatggtagttatagatcagaagagcttaatctagtaataataggaccctcagctgatattatacagtttgaatgtaaaagctttggacatgatgttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgccgaacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatgtagcaagtacacttaataaagctaaatcaataataggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaatttttttaaagtaattaacagaaaaacatatttgaattttgataaagccgtatttagaataaatatagtacctgatgaaaattacacaataaaggatggatttaatttaaaaggtgcaaatttatcaacaaattttaatggtcagaatacagaaattaatagtaggaattttactagattaaaaaattttacaggattatttgaattttataagctgctatgtgtaagagggataataccttttaaaactaaatcattagatgaaggatacaataag

SEQ ID NO: 5 - protein, 448 aa, synthetic construct
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDRLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 6 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDRLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNK

SEQ ID NO: 7 - DNA, 1344 bp, synthetic construct
  source 1..1344: mol_type = other DNA
atgccatttgttaataaacaatttaattataaagatcctgtaaatggtgttgatattgcttatataaaaattccaaatgtaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagatagattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataag

SEQ ID NO: 8 - DNA, 1344 bp, synthetic construct
  source 1..1344: mol_type = other DNA
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacctttacaaaccctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacagccagatggtagttatagatcagaagagcttaatctagtaataataggaccctcagctgatattatacagtttgaatgtaaaagctttggacatgatgttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgccgaacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatgtagcaagtacacttaataaagctaaatcaataataggtactactgcttcattacagtatatgaaaaatgtttttaaagagaaatatctcctatctgaagatacatctggaaaattttcggtagatagattaaaatttgataagttatacaaaatgttaacagagatttacacagaggataattttgttaatttttttaaagtaattaacagaaaaacatatttgaattttgataaagccgtatttagaataaatatagtacctgatgaaaattacacaataaaggatggatttaatttaaaaggtgcaaatttatcaacaaattttaatggtcagaatacagaaattaatagtaggaattttactagattaaaaaattttacaggattatttgaattttataagctgctatgtgtaagagggataataccttttaaaactaaatcattagatgaaggatacaataag

SEQ ID NO: 9 - DNA, 18 bp, synthetic construct
  source 1..18: mol_type = other DNA
ggtgcaggcagatttgct

SEQ ID NO: 10 - DNA, 18 bp, synthetic construct
  source 1..18: mol_type = other DNA
agcaaatctgcctgcacc

SEQ ID NO: 11 - DNA, 22 bp, synthetic construct
  source 1..22: mol_type = other DNA
ttttaaagagagatatctccta

SEQ ID NO: 12 - DNA, 22 bp, synthetic construct
  source 1..22: mol_type = other DNA
taggagatatctctctttaaaa

SEQ ID NO: 13 - DNA, 18 bp, synthetic construct
  source 1..18: mol_type = other DNA
catctggaagattttcgg

SEQ ID NO: 14 - DNA, 18 bp, synthetic construct
  source 1..18: mol_type = other DNA
ccgaaaatcttccagatg

SEQ ID NO: 15 - DNA, 22 bp, synthetic construct
  source 1..22: mol_type = other DNA
tttcggtagatagattaaaatt

SEQ ID NO: 16 - DNA, 22 bp, synthetic construct
  source 1..22: mol_type = other DNA
aattttaatctatctaccgaaa

SEQ ID NO: 17 - DNA, 21 bp, synthetic construct
  source 1..21: mol_type = other DNA
aatttgataggttatacaaaa

SEQ ID NO: 18 - DNA, 21 bp, synthetic construct
  source 1..21: mol_type = other DNA
ttttgtataacctatcaaatt

SEQ ID NO: 19 - DNA, 23 bp, synthetic construct
  source 1..23: mol_type = other DNA
actaaactaagaaattttactgg

SEQ ID NO: 20 - DNA, 23 bp, synthetic construct
  source 1..23: mol_type = other DNA
ccagtaaaatttcttagtttagt

SEQ ID NO: 21 - DNA, 48 bp, synthetic construct
  source 1..48: mol_type = other DNA
tcggtagatagattaaaatttgataagttatacaaaatgttaacagag

SEQ ID NO: 22 - DNA, 42 bp, synthetic construct
  source 1..42: mol_type = other DNA
atcaaattttaatctatctaccgaaaattttccagatgtatc

(19) United States
(12) Patent Application Publication
(10) Pub. No.: US 2026/0132395 A1
(43) Pub. Date: May 14, 2026

(54) LIGHT CHAIN MUTANT OF BOTULINUM TOXIN

(71) Applicant: ALKEMIER, INC., Seoul (KR)

(72) Inventors: Kwang-Hyun BAEK, Seoul (KR); Hae-Seul CHOI, Seongnam-si, Gyeonggi-do (KR); Myunghoon LEE, Seoul (KR)

(73) Assignee: ALKEMIER, INC., Seoul (KR)

(21) Appl. No.: 19/120,109

(22) PCT Filed: Oct. 10, 2023
(86) PCT No.: PCT/KR2023/015475

§ 371 Date: Apr. 10, 2025

(30) Foreign Application Priority Data
Oct. 12, 2022 (KR) 10-2022-0130397

(51) Int. Cl. C12N 9/52; C07K 14/33; C12N 15/85

(52) U.S. Cl. CPC C12N 9/52; C07K 14/33; C12N 15/85; C12Y 304/24069

(57) ABSTRACT

Provided is a light chain variant of botulinum toxin obtained by substituting a certain lysine with arginine in the light chain of botulinum toxin, which shows the suppression of degradation by the ubiquitin-proteasome system and thus a significantly increased in vivo half-life.

### TECHNICAL FIELD

The present invention relates to a variant of the light chain of botulinum toxin (also referred to as ‘a botulinum light chain protein’). More specifically, the present invention relates to a light chain variant of botulinum toxin, having an increased half-life, obtained by substituting a certain lysine with arginine in the light chain of botulinum toxin.

### BACKGROUND ART

Protein degradation in eukaryotic cells occurs through two pathways, i.e., by lysosomes and by proteasomes. The lysosomal pathway, which degrades 10% to 20% of proteins, lacks substrate specificity and precise temporal regulation. That is, it is a degrading process of most extracellular or membrane proteins, such as cell surface proteins incorporated into cells through endocytosis are degraded by lysosomes. However, selective degradation of proteins in eukaryotic cells requires the ubiquitin-proteasome pathway, which includes binding ubiquitin to a target protein by ubiquitin-conjugating enzyme, forming a polyubiquitin chain, and recognizing and degrading the polyubiquitin chain by proteasomes. More than 80% of eukaryotic proteins are degraded through this process, and thus the ubiquitin-proteasome pathway is responsible for functional transition and homeostasis of proteins through regulating the degradation of most proteins present in eukaryotic cells.

Botulinum toxin has a structure in which a heavy chain of about 100 kDa and a light chain of about 50 kDa are linked via disulfide bonds. The disulfide bonds play an important role in biological activation and action of the toxin and are easily cleaved by other surrounding factors due to their weak binding strength. The heavy chain consists of two functional terminals. It is known that the N-terminal portion acts as a translocation site to form an ion channel in the lipid bilayer, while the C-terminal portion acts as a binding site to play an important role in internalization through binding the toxin to the cell membrane. The light chain acts as a zinc-dependent endopeptidase. Botulinum toxin is mostly used for facial cosmetic purposes. It is used by injecting the toxin into the skin to improve wrinkles on the forehead and between the eyebrows. And, it is also used in the treatment of diseases due to its benefits such as paralysis of nerves therethrough. However, these usually lasts for about 6 months, after which the skin and disease site return to their original shape. Continuous injection of botulinum toxin may induce tolerance and the side effects therefrom cannot be excluded. Therefore, there is a need for a method that can maintain the effects thereof while injecting the smallest amount of toxin.

### DETAILED DESCRIPTION OF THE INVENTION

### Technical Problem

The present inventors performed various studies to develop a method capable of effectively increasing the in vivo half-life of botulinum toxin to allow long-term treatment or small dose-used treatment. The present inventors found that the light chain of botulinum toxin undergoes the degradation pathway via ubiquitin-proteasome. And the present inventors prepared various variants to compare the ubiquitination-degradation pathway thereof. As a result, it has been found that a variant obtained by substituting a certain lysine (i.e., lysine at position 335) with arginine in the light chain of botulinum toxin shows significant suppressions in ubiquitination-degradation.

Therefore, it is an object of the present invention to provide a light chain variant of botulinum toxin obtained by substituting a certain lysine (i.e., lysine at position 335) with arginine.

It is another object of the present invention to provide a vector comprising a gene encoding the light chain variant of botulinum toxin.

It is still another object of the present invention to provide a cell transfected with a vector comprising a gene encoding the light chain variant of botulinum toxin.

It is still another object of the present invention to provide a method for increasing the half-life of a light chain of botulinum toxin, the method comprising substituting a certain lysine (i.e., lysine at position 335) with arginine, in the light chain of botulinum toxin.

### Technical Solution

In accordance with an aspect of the present invention, there is provided a light chain variant of botulinum toxin wherein lysine at position 335 in the light chain of botulinum toxin consisting of the amino acid sequence of SEQ ID NO: 1 or 2 is substituted with arginine.

In accordance with another aspect of the present invention, there is provided a vector comprising a gene encoding the light chain variant of botulinum toxin.

In accordance with still another aspect of the present invention, there is provided a cell transfected with a vector comprising a gene encoding the light chain variant of botulinum toxin.

In accordance with still another aspect of the present invention, there is provided a method for increasing the half-life of a light chain of botulinum toxin, the method comprising substituting lysine at position 335 with arginine, in the light chain of botulinum toxin consisting of the amino acid sequence of SEQ ID NO: 1 or 2.

### Advantageous Effects

The light chain variants of botulinum toxin according to the present invention show significant suppressions in degradation by the ubiquitin-proteasome system and thus have a significantly increased in vivo half-life. Therefore, the light chain variants of botulinum toxin according to the present invention may be usefully used for preparing a botulinum toxin that allows long-term treatment or small dose-used treatment.

### BRIEF DESCRIPTION OF THE DRAWINGS

FIG. 1a shows the results obtained by confirming the expression after inducing transfection while increasing the amount of plasmid gene for the light chain of botulinum toxin type A1 in the B16F10 cell line.

FIG. 1b shows the results obtained by confirming the expression after inducing transfection while increasing the amount of plasmid gene for the light chain of botulinum toxin type A2 in the HeLa cell line.

FIG. 2a shows the results obtained by confirming the degradation pathway of the light chain of botulinum toxin type A1 through ubiquitination analysis.

FIG. 2b shows the results obtained by confirming the degradation pathway of the light chain of botulinum toxin type A2 through ubiquitination analysis.

FIG. 3a shows the results obtained by comparing the ubiquitination levels of the light chain protein of wild-type botulinum toxin type A1 and the light chain protein variants of botulinum toxin type A1.

FIG. 3b shows the results obtained by comparing the ubiquitination levels of the light chain protein of wild-type botulinum toxin type A2 and the light chain protein variant of botulinum toxin type A2.

FIG. 4a shows the results obtained by comparing the stabilization levels of the light chain of botulinum toxin type A1 in cells after the treatment with cycloheximide.

FIG. 4b shows the results obtained by comparing the stabilization levels of the light chain of botulinum toxin type A2 in cells after the treatment with cycloheximide.

FIG. 5a shows a graph that numerically represents the results of FIG. 4a (*: 0.01<p<0.05, ns: p>0.05).

FIG. 5b shows a graph that numerically represents the results of FIG. 4b (*: 0.01<p<0.05, ns: p>0.05).

### BEST MODE FOR CARRYING OUT THE INVENTION

The present inventors confirmed that the light chain of botulinum toxin undergoes the degradation pathway via ubiquitin-proteasome. And, the present inventors, through site-directed mutagenesis, prepared conservative amino acid substitution variants for the light chain protein of botulinum toxin type A1, i.e. the variants wherein lysine at position 212, 320, 330, 335, 340, or 417 in the light chain protein of botulinum toxin type A1 was substituted with arginine; and then evaluated the degradation levels by ubiquitin-proteasome. In addition, the present inventors prepared a conservative amino acid substitution variant for the light chain protein of botulinum toxin type A2, i.e. the variant wherein lysine at position 335 in the light chain protein of botulinum toxin type A2 was substituted with arginine; and then evaluated the degradation level by ubiquitin-proteasome. The present inventors have found that the variants obtained by substituting lysine at position 335 with arginine (i.e., the light chain variants of botulinum toxin consisting of the amino acid sequence of SEQ ID NO: 5 or 6) exhibit significant suppressions in degradation by the ubiquitin-proteasome and thus has a significantly increased in vivo half-life. Therefore, said variants may be usefully used for preparing a botulinum toxin that allows long-term treatment or small dose-used treatment.

The present invention provides a variant of the light chain protein of botulinum toxin. That is, the present invention provides a light chain variant of botulinum toxin wherein lysine at position 335 is substituted with arginine, in the light chain of botulinum toxin type A1 consisting of the amino acid sequence of SEQ ID NO: 1 or in the light chain of botulinum toxin type A2 consisting of the amino acid sequence of SEQ ID NO: 2.

Both the amino acid sequence of the light chain protein of botulinum toxin and the base sequence encoding the same are known in the art. The amino acid sequence of the light chain protein of botulinum toxin type A1 is as in SEQ ID NO: 1 and the base sequence encoding the same is as in SEQ ID NO: 3. And, the amino acid sequence of the light chain protein of botulinum toxin type A2 is as in SEQ ID NO: 2 and the base sequence encoding the same is as in SEQ ID NO: 4.

In an embodiment, the light chain variant of botulinum toxin according to the present invention may be a variant consisting of the amino acid sequence of SEQ ID NO: 5 or 6.

The light chain variants of botulinum toxin according to the present invention may be prepared by substituting lysine at position 335 with arginine in the light chain of botulinum toxin consisting of the amino acid sequence of SEQ ID NO: 1 or 2, according to a method conventionally used in the field of biotechnology.

For example, using a gene encoding the light chain protein of botulinum toxin type A1 (e.g., a gene having the base sequence of SEQ ID NO: 3) as a template, polymerase chain reactions may be carried out with the primer sets of SEQ ID NOS: 15 and 16 below, so as to obtain a gene encoding the variant in which lysine at position 335 is substituted with arginine. In an embodiment, the gene encoding the variant in which lysine at position 355 is substituted with arginine may consist of the base sequence of SEQ ID NO: 7.

In addition, for example, using a gene encoding the light chain protein of botulinum toxin type A2 (e.g., a gene having the base sequence of SEQ ID NO: 4) as a template, polymerase chain reactions may be carried out with the primer sets of SEQ ID NOs: 21 and 22 below, so as to obtain a gene encoding the variant in which lysine at position 335 is substituted with arginine. In another embodiment, the gene encoding the variant in which lysine at position 355 is substituted with arginine may consist of the base sequence of SEQ ID NO: 8.

The obtained genes may be used for preparing an expression vector according to a conventional method used in the field of biotechnology, followed by transfecting a host cell to obtain a transfected cell. The transfected cell may be cultured to obtain said variant.

Therefore, the present invention includes a vector (i.e., an expression vector) comprising a gene encoding the light chain variant of botulinum toxin. The gene may consist of the base sequence of SEQ ID NO: 7 or 8. The expression vector may be prepared using a vector conventionally used in the field of biotechnology, such as pcDNA3, pCS4, pcDNA3.1, etc., as an empty vector, with an appropriate restriction enzyme. The empty vector may be a vector labeled with a flag, etc., if necessary.

In addition, the present invention includes a cell transfected with a vector (i.e., an expression vector) comprising a gene encoding the light chain variant of botulinum toxin. The gene may consist of the base sequence of SEQ ID NO: 7 or 8. Host cells include, but are not limited to, HEK293T cells, B16F10 cells, A549 cells, A2780 cells, SKOV3 cells, Hela cells, etc.

The present invention also provides a method for increasing the half-life of a light chain of botulinum toxin, the method comprising substituting lysine at position 335 with arginine, in the light chain of botulinum toxin consisting of the amino acid sequence of SEQ ID NO: 1 or 2. In the method of the present invention, the substitution is as described above.

Hereinafter, the present invention will be described more specifically by the following examples. However, the following examples are provided only for illustrations and thus the present invention is not limited to or by them.

### EXAMPLES

### 1. Experimental Method

### (1) Construction of Expression Vectors

The polynucleotide of the light chain of botulinum toxin type A1 consisting of the base sequence of SEQ ID NO: 3 was cloned into the pCS4-3Flag vector (4.3 kb) (E7908, Sigma-Aldrich) using the restriction enzymes EcoRI and XhoI to construct an expression vector (pCS4-3Flag-Bont-LC WT) of the light chain protein of wild-type botulinum toxin type A1. And, a polynucleotide of the light chain of botulinum toxin type A2 consisting of the base sequence of SEQ ID NO: 4 was cloned into the pCS4-3Flag vector (4.3 kb) (E7908, Sigma-Aldrich) using the restriction enzymes EcoRI and XhoI to construct an expression vector of the light chain protein of wild-type botulinum toxin type A2 (pCS4-3Flag-Bont-A2-LC WT).

Using the primer sets in Table 1 below, we prepared expression vectors for the variants in which lysine at position 212, 320, 330, 335, 340, or 417 was respectively substituted with arginine in the light chain protein of botulinum toxin type A1; and an expression vector for the variant in which lysine at position 335 was substituted with arginine in the light chain protein of botulinum toxin type A2, through site-directed mutagenesis. Specifically, using the above-cloned pCS4-3Flag-Bont-LC WT gene for the light chain protein of wild-type botulinum toxin type A1 as a template, polymerase chain reactions (PCRs) were performed with each primer set. The polymerase chain reactions (PCRs) were performed under the following conditions: 12 cycles in total: 95° C. for 30 seconds, 60° C. for 30 seconds, and 68° C. for 5 minutes and 40 seconds. Through the mutagenesis, we constructed the six expression vectors for each variant of the light chain of botulinum toxin type A1, i.e., pCS4-3Flag-Bont-LC (K212R), pCS4-3Flag-Bont-LC (K320R), pCS4-3Flag-Bont-LC (K330R), pCS4-3Flag-Bont-LC (K335R), pCS4-3Flag-Bont-LC (K340R), and pCS4-3Flag-Bont-LC (K417R). In addition, using the above-cloned pCS4-3Flag-Bont-A2-LC WT gene for the light chain protein of wild-type botulinum toxin type A2 as a template, polymerase chain reactions (PCR) was performed with the primer set SEQ ID NOs: 21 and 22. The polymerase chain reaction (PCR) was performed under the following conditions: 18 cycles in total: 98° C. for 10 seconds, 58° C. for 5 seconds, and 72° C. for 5 minutes and 40 seconds. We constructed the expression vector for the variant of the light chain of botulinum toxin type A2, i.e., pCS4-3Flag-Bont-A2-LC (K335R).

TABLE 1

| | SEQ | | |
|---|---|---|---|
| Expression Vector | ID NO | Sequence | |
| pCS4-3Flag-Bont-LC | 9 | Forward | 5′-GGT GCA GGC AGA TTT GCT-3′ |
| (K212R) | 10 | Reverse | 5′-AGC AAA TCT GCC TGC ACC-3′ |
| pCS4-3Flag-Bont-LC | 11 | Forward | 5′-TTT TAA AGA GAG ATA TCT CCT |
| (K320R) | | | A-3′ |
| | 12 | Reverse | 5′-TAG GAG ATA TCT CTC TTT AAA |
| | | | A-3′ |
| pCS4-3Flag-Bont-LC | 13 | Forward | 5′-CAT CTG GAA GAT TTT CGG-3′ |
| (K330R) | 14 | Reverse | 5′-CCG AAA ATC TTC CAG ATG-3′ |
| pCS4-3Flag-Bont-LC | 15 | Forward | 5′-TTT CGG TAG ATA GAT TAA AAT |
| (K335R) | | | T-3′ |
| | 16 | Reverse | 5′-AAT TTT AAT CTA TCT ACC GAA |
| | | | A-3′ |
| pCS4-3Flag-Bont-LC | 17 | Forward | 5′-AAT TTG ATA GGT TAT ACA AAA-3′ |
| (K340R) | 18 | Reverse | 5′-TTT TGT ATA ACC TAT CAA ATT-3′ |
| pCS4-3Flag-Bont-LC | 19 | Forward | 5′-ACT AAA CTA AGA AAT TTT ACT |
| (K417R) | | | GG-3′ |
| | 20 | Reverse | 5′-CCA GTA AAA TTT CTT AGT TTA |
| | | | GT-3′ |
| pCS4-3Flag-Bont-A2- | 21 | Forward | 5′-TCG GTA GAT AGA TTA AAA TTT |
| LC (K335R) | | | GAT AAG TTA TAC AAA ATG TTA ACA |
| | | | GAG-3′ |
| | 22 | Reverse | 5′-ATC AAA TTT TAA TCT ATC TAC |
| | | | CGA AAA TTT TCC AGA TGT ATC-3′ |

### (2) Transfection

Transfections were induced into B16F10 cells (ATCC, CRL-6475), using the expression vectors, i.e., pCS4-3Flag-Bont-LC (K212R), pCS4-3Flag-Bont-LC (K320R), pCS4-3Flag-Bont-LC (K330R), pCS4-3Flag-Bont-LC (K335R), pCS4-3Flag-Bont-LC (K340R), and pCS4-3Flag-Bont-LC (K417R), respectively. And, transfection was induced into Hela cells (ATCC, CCL-2), using the expression vector pCS4-3Flag-Bont-A2-LC (K335R).

B16F10 cells and Hela cells were cultured in Dulbecco's Modified Eagle's Medium (DMEM, Gibco, Grand Island, NY, USA) containing 10% fetal bovine serum (FBS, Gibco, Grand Island, NY, USA) and 1% penicillin and streptomycin (Gibco, Grand Island, NY, USA) in a 5% CO2 incubator. For transfection of botulinum toxin type A1 light chain WT, K212R, K320R, K330R, K335R, K340R, or K417R and ubiquitin pRK5-HA-Ub; or for transfection of botulinum toxin type A2 light chain WT or K335R and ubiquitin pRK5-HA-Ub, 3 μg of the wild type or the variant of the light chain of botulinum toxin type A1 or A2, 3 μg of ubiquitin, 600 μL of NaCl, and 42 μL of polyethylenimine reagent (PEI; Polysciences, Inc., Warrington, PA, USA) were mixed and reacted at room temperature for 15 minutes. The mixture was added to 6 mL of the culture medium containing B16F10 cells (1×10⁶ cells) or Hela cells (1×10⁶ cells) and then cultured at 37° C. for 48 hours.

### (3) Immunoblotting and Antibody

Immunoblotting was performed using anti-Flag antibody (MBL), anti-HA antibody (12CA5 hybridoma cell media), and anti-β-actin antibody (Santa Cruz Biotechnology). After SDS-PAGE, the membrane was transferred to a polyvinylidene difluoride (PVDF) membrane (Millipore) and then blot detection was performed using an HRP-conjugated secondary antibody.

### (4) Immunoprecipitation

Transfected cells were lysed in a lysis buffer (50 mM Tris-HCl [pH 7.5], 1 mM EDTA, 10% glycerol, 300 mM NaCl, and 1% Triton X-100) on ice for 20 min, and then centrifuged at 13,000 rpm for 20 min. The supernatant was taken and the antibody (Flag antibody) was added thereto. The mixture was reacted overnight at 4° C. and then the A/G PLUS agarose beads (Santa Cruz Biotechnology, Santa Cruz, CA, USA) were added thereto. The mixture was reacted for 2 hours on a 4° C. rotator to obtain only the Flag-labeled botulinum toxin type A1 light chain (Flag-Bont-LC) and the Flag-labeled botulinum toxin type A2 light chain (Flag-Bont-A2-LC) among the proteins expressed in the B16F10 cells or the Hela cells. Antibodies, beads, and proteins were mixed with a 2×SDS (sodium dodecyl sulfate) buffer, boiled at 100° C. for 7 minutes to break the bonds, and structural unfolding of the light chain of botulinum toxin was induced, followed by separation by SDS-PAGE. The separated proteins were transferred to a polyvinylidene difluoride membrane, which was incubated overnight at 4° C. with primary antibodies [anti-Flag (MBL), anti-HA (12CA5 hybridoma cell media), and anti-β-actin (Santa Cruz Biotechnology) antibodies] mixed with 2% skim milk. The blot was then developed onto a photosensitive film with an enhanced chemiluminescence (ECL) system, using an anti-mouse secondary monoclonal antibody.

### (5) Confirmation of the Results and Statistical Analysis

Densitometric analysis was performed using Image J (National Institutes of Health), and Turkey test was performed using GraphPad Prism version 5 (GraphPad Software). ANOVA was performed in a one-way analysis to show significant differences.

### 2. Experimental Results

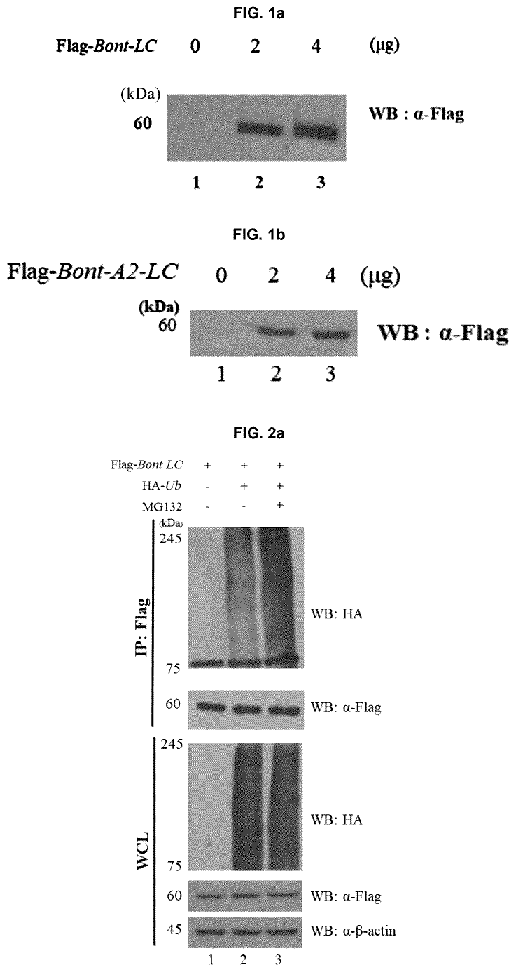

Agarose gel electrophoresis was performed on the cells transfected with the expression vectors for the light chain protein of wild-type botulinum toxin type A1 or A2. Only Flag-Bont-LC or Flag-Bont-A2-LC was specifically isolated using the antibodies, and transfection was performed while increasing the amount of plasmid gene to confirm the exact protein size. Flag-Bont-LC and Flag-Bont-A2-LC were confirmed to have a size of approximately 51-54 kDa; and it is also confirmed that their expressions were induced in the cell lines (FIG. 1a and FIG. 1b).

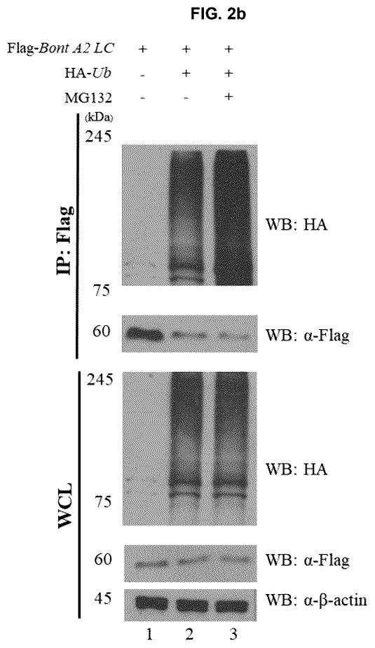

For analysis of ubiquitination of the light chain of botulinum toxin, B16F10 cells were transfected with the pCS4-3Flag-Bont-LC WT and the pRK5-HA-Ub plasmid gene; and HeLa cells were transfected with the pCS4-3Flag-Bont-A2-LC WT and the pRK5-HA-Ub plasmid gene. The light chain of botulinum toxin transfected into the cell line was precipitated by immunoprecipitation analysis to determine the levels of ubiquitination. As a result of the treatments with the MG132 reagent, the level of ubiquitination increased, thereby confirming that the light chains of botulinum toxin types A1 and A2 undergo the degradation pathway via ubiquitin-proteasome (FIG. 2a and FIG. 2b).

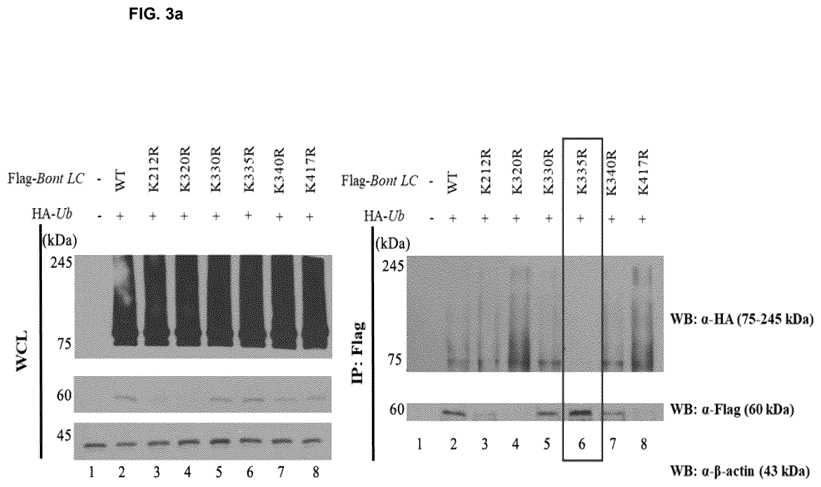

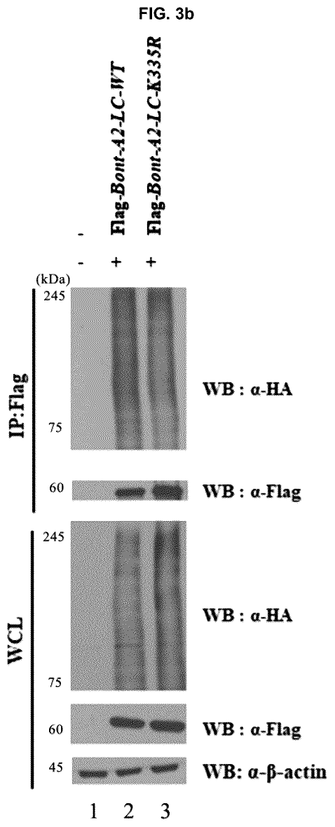

Transfection of B16F10 cells was induced using pCS4-3Flag-Bont-LC WT, pCS4-3Flag-Bont-LC (K212R), pCS4-3Flag-Bont-LC (K320R), pCS4-3Flag-Bont-LC (K330R), pCS4-3Flag-Bont-LC (K335R), pCS4-3Flag-Bont-LC (K340R), pCS4-3Flag-Bont-LC (K417R), and pRK5-HA-Ub plasmid genes, and the levels of ubiquitination was compared as in above. Additionally, transfection of Hela cells was induced using pCS4-3Flag-Bont-A2-LC WT, pCS4-3Flag-Bont-A2-LC (K335R), and pRK5-HA-Ub plasmid gene, and the levels of ubiquitination were compared as in above. As a result, in the case of the variants in which lysine at position 335 was substituted with arginine, the levels of ubiquitination were significantly reduced compared to the control group (FIG. 3a and FIG. 3b).

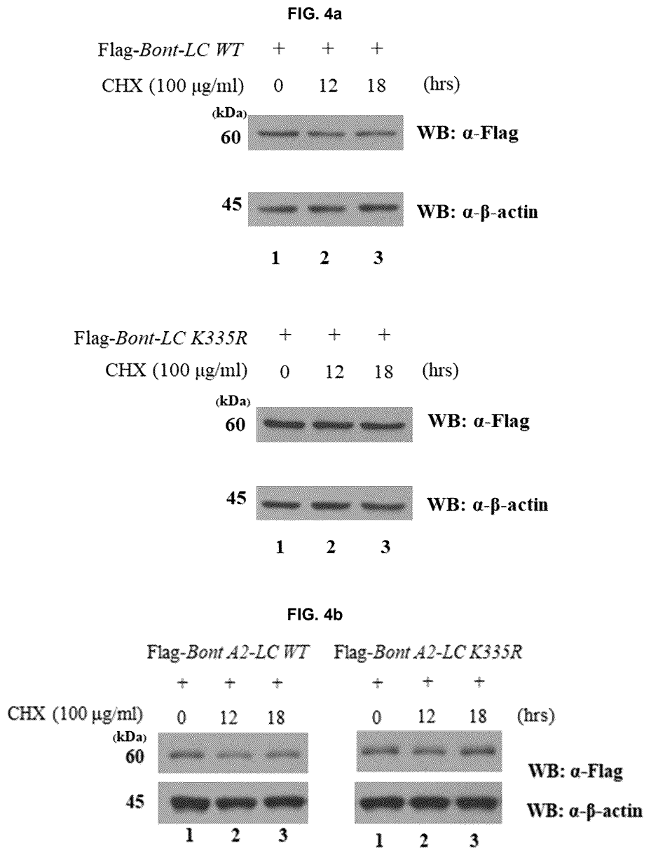

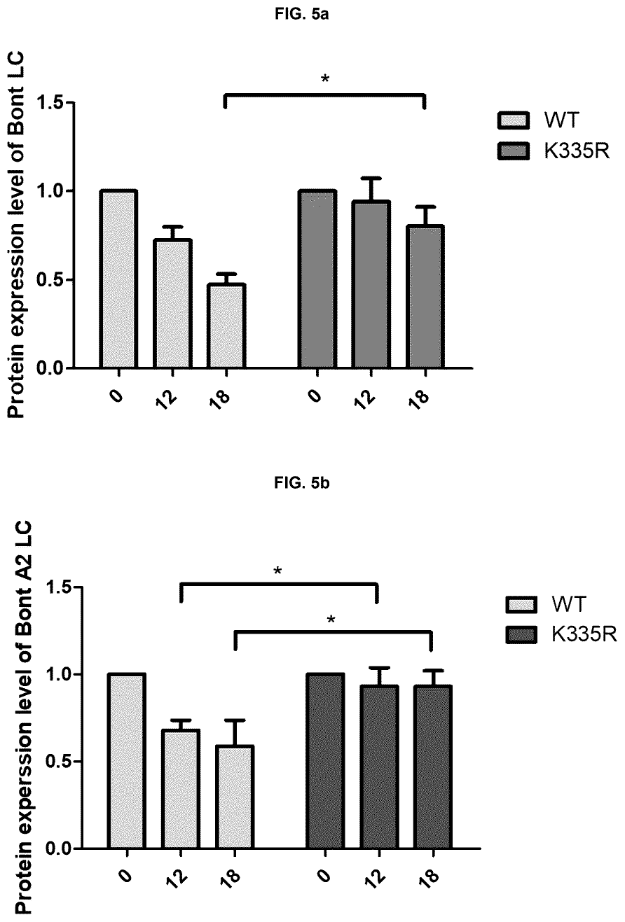

B16F10 cells were transfected with the same amount of pCS4-3Flag-Bont-LC WT and pCS4-3Flag-Bont-LC (K335R) plasmid genes, and after 24 hours therefrom, cycloheximide (CHX) was treated in each cell medium at a concentration of 100 μg/mL for 0 hour, 12 hours, and 18 hours, and then immunoblotting was performed. The results are as shown in FIG. 4a. In addition, Hela cells were transfected with the same amount of pCS4-3Flag-Bont-A2-LC WT and pCS4-3Flag-Bont-A2-LC (K335R) plasmid genes, and after 48 hours therefrom, cycloheximide (CHX) was treated in each cell medium at a concentration of 100 μg/ml for 0 hour, 12 hours, and 18 hours, and then immunoblotting was performed. The results are as shown in FIG. 4b. FIG. 5a and FIG. 5b are the graphs that numerically represent the results of FIG. 4a and FIG. 4b, respectively. From the results in FIG. 4a and FIG. 5a, it can be seen that the light chain variant of botulinum toxin type A1 with a substitution of the lysine residue at position 335 showed a significant increase in protein stability (i.e., 1.69-fold at 18 hours). In addition, from the results in FIG. 4b and FIG. 5b, it can be seen that the light chain variant of botulinum toxin type A2 with a substitution of the lysine residue at position 335 also showed a significant increase in protein stability (i.e., 1.24-fold at 18 hours).

1. A light chain variant of botulinum toxin wherein lysine at position 335 in the light chain of botulinum toxin consisting of the amino acid sequence of SEQ ID NO: 1 or 2, is substituted with arginine.

2. The light chain variant of botulinum toxin according to claim 1, consisting of the amino acid sequence of SEQ ID NO: 5 or 6.

3. A vector comprising a gene encoding the light chain variant of botulinum toxin according to claim 1.

4. The vector according to claim 3, wherein the gene consists of the base sequence of SEQ ID NO: 7 or 8.

5. A cell transfected with a vector comprising a gene encoding the light chain variant of botulinum toxin according to claim 1.

6. The cell according to claim 5, wherein the gene consists of the base sequence of SEQ ID NO: 7 or 8.

7. A method for increasing the half-life of a light chain of botulinum toxin, the method comprising substituting lysine at position 335 with arginine, in the light chain of botulinum toxin consisting of the amino acid sequence of SEQ ID NO: 1 or 2.

8. A vector comprising a gene encoding the light chain variant of botulinum toxin according to claim 2.

9. The vector according to claim 8, wherein the gene consists of the base sequence of SEQ ID NO: 7 or 8.

10. A cell transfected with a vector comprising a gene encoding the light chain variant of botulinum toxin according to claim 2.

11. The cell according to claim 10, wherein the gene consists of the base sequence of SEQ ID NO: 7 or 8.
